(19) United States
(12) Patent Application Publication
(10) Pub. No.: US 2009/0023998 A1
(43) Pub. Date: Jan. 22, 2009

(54) REAR VIEW ENDOSCOPE SHEATH

(76) Inventor: Nitesh Ratnakar, Elkins, WV (US)

Correspondence Address: BOYLE FREDRICKSON S.C., 840 North Plankinton Avenue, MILWAUKEE, WI, 53203, US

(21) Appl. No.: 11/778,987

(22) Filed: Jul. 17, 2007

(51) Int. Cl. A61B 1/00

(52) U.S. Cl. 600/121

(57) ABSTRACT

The present invention relates to an endoscope sheath, more specifically to an endoscope sheath that provides rear view of a hollow body organ when used in conjunction with a corresponding endoscope at the same time when the corresponding endoscope provides forward view of the hollow organ. The endoscope sheath is sized to slip over a corresponding endoscope. It comprises of a rear view module containing a rear image lens and a rear illumination bulb. The rear view module is designed and is attached to the endoscope sheath in a way that when deployed, the rear image lens and the rear illumination bulb face backward with respect to the forward axis of the corresponding endoscope. In this position, the rear image lens of the endoscope sheath provides a rear view while the main image lens of the endoscope provides forward view. The ability to obtain forward and rear view at the same time enables the operator to perform a complete examination of a hollow organ in a single insertion.

### FIELD OF INVENTION

The present invention relates to endoscope sheath, more specifically to endoscope sheath with an integrated rear view module with means to provide rear view during endoscopic examination of a hollow body organ.

### BACKGROUND AND PRIOR ART

Endoscopes are used to perform a variety of surgical procedures. FIGS. 1 and 2 illustrate an embodiment of a conventional endoscope. It has a handle from which extends a flexible shaft, which is inserted into a hollow organ to be inspected. The shaft consists of a proximal section, insertion tube, bending section and a stiff section. The shaft terminates in the distal end, which typically houses image lens, illumination bulb, air/water nozzle and an instrument channel outlet. Light is transmitted from a light source through the shaft via an electric cable to the illumination bulb. The illumination bulb illuminates the area to be examined. The image lens captures images of the illuminated area. The image is then transmitted through a fiber optic cable and viewed through an eyepiece on the handle of the endoscope. Alternatively, the image is converted to a video signal and transmitted to an image processor by an electrical cable. The image is then processed and displayed on a display unit like a computer monitor. The handle of the endoscope has an extension arm that attaches the endoscope to a light source and an image processor.

To enable the endoscope to maneuver through the turns of a hollow organ the shaft is flexible and incorporates a multitude of cables that attach the bending portion with actuators. Tension is applied to these cables to move the bending portion in various directions. This is done by manual adjustment of actuators on the handle of the endoscope. Typically, there are two pairs of such cables passing within the shaft, one pair for flexing the bending portion in one plane and the other pair for flexing it in an orthogonal plane.

It is also usual to provide two channels extending between the handle and the distal end of the shaft, an air/water channel and an instrument channel. The air/water channel is used to insufflate air in a hollow organ to expand it for proper visualization. The air/water channel is connected proximally to an air/water pump (not shown) and to distally to the air/water channel outlet. The image lens and the illumination bulb are frequently smeared with blood, stool or other body fluids while in a hollow organ which obstructs a clear view. In such a situation, the air/water channel is used to eject water or blow air at the image lens and/or illumination bulb in order to clean them while still inside a hollow organ. The instrument channel has an inlet proximally and an outlet distally. It is used to pass various surgical instruments to do various surgical procedures. It is also used to apply suction to remove fluids, air and other materials from within a hollow organ during examination.

Endoscope is typically inserted into the patient either thorough a natural body orifice like anus or mouth or it is inserted through a surgical incision. It is then steered to a desired location by adjusting the bending portion and manually pushing the endoscope. After reaching the desired location, the endoscope is withdrawn. Typically it is during pullout when the inside of a hollow organ like colon is thoroughly examined. Insertion of the endoscope into a hollow organ is a risky maneuver and is associated with significant complications like trauma, bleeding and perforation. It is generally desirable to complete the examination with a single insertion to minimize complications.

The present endoscopes have significant limitations. As shown in FIG. 3 they are only forward viewing. Currently, rear view can only be obtained by bending the distal portion of the endoscope back upon itself in a ‘retro flexion’ maneuver as shown in FIG.4B. However, it is not possible to achieve retro flexion in many narrow hollow organs like colon, esophagus, duodenum and small bowel. Also, retro flexion compromises forward view. Hence with conventional endoscopes, only one view, forward or backward, is possible at a given time. The present endoscopes also have a narrow field of vision with an angle of vision of about 120 degrees. A large number of significant pathologic findings are frequently missed during endoscopic examination because the inability to obtain rear view and a narrow field of vision of conventional endoscopes.

This is especially true for colonoscopy where the inside of the colon is examined with an endoscope. Many cancers and pre cancerous lesions (polyps) are frequently missed during colonoscopy (Pickhardt P J, Choi J R, Hwang I, Butler J A, Puckett M L, Hildebrandt H A, Wong R K, Nugent P A, Mysliwiec P A, Schindler W R. Computed tomographic virtual colonoscopy to screen for colorectal neoplasia in asymptomatic adults; N Engl J Med. Dec. 4, 2003; 349(23):2191-200). This has serious consequences including death, many of which can easily be prevented. Majority of the missed lesions lie on the rear side of mucosal folds (Pickhardt P J, Nugent P A, Mysliwiec P A, Choi J R, Schindler W R. Location of adenomas missed by optical colonoscopy. Ann Intern Med. Sep. 7, 2004; 141(5):352-9). With forward viewing endoscopes, the front of mucosal folds obstructs visualization of the rear side as shown in FIG. 4A. Currently, the rear side of a mucosal fold can only be examined by pushing the tip of the endoscope beyond the fold and bending the endoscope back upon itself in a ‘retro flexion’ maneuver as shown in FIG. 4B. However, it is frequently not possible to achieve retro flexion in a narrow hollow organ like colon.

Also, retro flexion maneuver compromises the forward view. With conventional endoscopes, only one view, forward or backward, is possible at a given time. Complete examination of colon that includes both forward and rear views currently requires multiple insertions, one to obtain forward view and other to obtain backward view by retro flexion. Both, retro flexion and multiple insertions, independently increase the morbidity, mortality, time and cost of colonoscopy. Moreover, intra colonic retro flexion can not be obtained frequently because of a narrow colonic lumen.

### SUMMARY OF THE INVENTION

In light of the significant limitations discussed above, there is a need for an endoscopic system that provides both forward and rear view during examination of a hollow body organ. The present invention enables rear view even in organs with a narrow lumen without the need to retro flex the endoscope. This is achieved by an endoscope sheath designed to sleeve over a corresponding endoscope; and containing a suitably designed ‘rear view module’. The rear view module consists of a rear image lens connected to an image processor and a rear illumination bulb connected to a light source. The rear view module can be of different shapes, size and configurations as illustrated in the embodiments of the present invention. The rear view module is attached to the endoscope sheath using one of many available methods of articulation as illustrated in the embodiments of the present invention. In the preferred method of examination of a hollow body organ, the endoscope sheath is drawn over an endoscope following which the endoscope is inserted into a hollow body organ. Once in the hollow organ, the rear view module on the endoscope sheath is deployed, which positions the rear view module facing backwards relative to the long axis of the endoscope thereby providing rear view of the hollow organ. The main image lens of the endoscope provides forward view. Means is provided for the operator to obtain forward and rear views either simultaneously or separately. This has the advantage of allowing a thorough examination that includes both forward and rear views in a single passage through a hollow body organ. Additional features and advantages of the present invention will be set forth in the description and drawings which follow or may be learned by practice of the invention.

### BRIEF DESCRIPTION OF THE DRAWINGS

FIG. 1 shows a side view of a conventional endoscope;

FIG. 2 shows a side view of the distal end, bending section and insertion tube of a conventional endoscope;

FIG. 3 is a side view of a conventional endoscope displaying the field of vision of a conventional endoscope;

FIG. 4A shows a conventional endoscope inside a colon. It shows mucosal folds of the colon and illustrates that visualization of the area behind mucosal folds is obstructed by the front of the mucosal folds during examination with a conventional endoscope;

FIG. 4B shows a conventional endoscope inside a colon in a retroflexed position. It displays how retro flexion enables visualization of area behind a mucosal fold;

FIG. 5 shows side view of an endoscope sheath;

FIG. 6A shows the first preferred configuration of the endoscope sheath wherein the endoscope sheath is positioned over the distal end of the endoscope. The pull wires and electrical wires from the rear view module stretches over the shaft of the endoscope;

FIG. 6B shows the second preferred configuration of the wherein the endoscope sheath is positioned over the distal end of the endoscope. The pull wires and electrical wires from the rear view module traverses through the instrument channel or a dedicated separate channel in the endoscope;

FIG. 6C shows the third preferred configuration of the wherein the endoscope sheath is positioned over the entire length of the endoscope. The pull wires and electrical wires from the rear view module traverses through the endoscope sheath;

FIGS. 7A, 7B and 7C show actuation means for rear view module contained in the embodiment of the endoscope sheath shown in FIGS. 8 and 9;

FIGS. 7D, 7E and 7F show actuation means for rear view module contained in the embodiment of the endoscope sheath shown in FIGS. 10, 11, 14 and 15;

FIGS. 7G, 7H and 7I show actuation means for rear view module contained in the embodiment of the endoscope sheath shown in FIGS. 23, 24, 27 and 28;

FIG. 8 shows the first preferred embodiment of the rear view endoscope sheath where the rear view module is attached sideways to the shaft of the endoscope sheath;

FIG. 9 shows side view of the endoscope sheath in FIG. 8 wherein the ‘rear view module is deployed for rear view;

FIG. 10 shows side view of an endoscope sheath with a ‘rear view module’ according to a second preferred embodiment of the present invention;

FIG. 11 is a side view of the endoscope sheath in FIG. 10 wherein the ‘rear view module’ is deployed for rear view;

FIG. 12 shows side view of an endoscope sheath with a ‘rear view module’ according to a third embodiment of the present invention.

FIG. 13 shows side view of the endoscope sheath in FIG. 12 wherein the ‘rear view module’ is deployed for rear view;

FIG. 14 shows side view of an endoscope sheath with a ‘rear view module’ according to a fourth embodiment of the present invention.

FIG. 15 shows side view of the endoscope sheath in FIG. 14 wherein the ‘rear view module’ is deployed for rear view;

FIG. 16 shows side view of an endoscope sheath with a ‘rear view module’ according to fifth embodiment of the present invention;

FIG. 17 is a side view of the endoscope sheath in FIG. 16 wherein the ‘rear view module’ is deployed for rear view;

FIG. 18 shows side view of an endoscope sheath with a ‘rear view module’ according to sixth embodiment of the present invention;

FIG. 19 is a side view of the endoscope sheath in FIG. 18 wherein the ‘rear view module’ is deployed for rear view;

FIG. 20 shows the second preferred configuration of the rear view module where it is attached to the front side of the endoscope as illustrated in embodiments of the invention shown in FIGS. 21-24;

FIG. 21A shows side view of an endoscope sheath with a ‘rear view module’ according to seventh embodiment of the present invention;

FIG. 21B shows a bi-planar joint mechanism used in endoscope sheath in embodiments shown in FIGS. 21A, 22, 29 and 30;

FIG. 21 C shows the actuation means of the rear view module when attached to the endoscope sheath by means of bi-planar joint, as in embodiments shown in FIGS, 21A, 22, 29 and 30;

FIG. 22 is a side view of the endoscope sheath in FIG. 21 wherein the ‘rear view module’ is deployed for rear view;

FIG. 23 shows side view of an endoscope sheath with a ‘rear view module’ according to eighth embodiment of the present invention;

FIG. 24 is a side view of the endoscope sheath in FIG. 23 wherein the ‘rear view module’ is deployed for rear view;

FIGS. 25A and 25B show spring mechanism as actuation means for the rear view module in the second preferred configuration of the rear view module where it is attached to the front side of the endoscope sheath as illustrated in FIG. 20 and in embodiments of the invention shown in FIGS. 21-24;

FIG. 26 shows the third preferred configuration of the rear view module where it is attached to the front side of the endoscope sheath as illustrated in embodiments of the invention shown in FIGS. 27-30;

FIG. 27 shows side view of an endoscope sheath with a ‘rear view module’ according to a ninth embodiment of the present invention;

FIG. 28 is a side view of the endoscope sheath in FIG. 27 wherein the ‘rear view module’ is deployed for rear view;

FIG. 29 shows side view of an endoscope sheath with a ‘rear view module’ according to the tenth embodiment of the present invention;

FIG. 30 is a side view of the endoscope sheath in FIG. 29 wherein the ‘rear view module’ is deployed for rear view; and

FIGS. 31A and 31B show spring mechanism as actuation means for the rear view module in the third preferred configuration of the rear view module where it is attached to the front side of the endoscope sheath as illustrated in FIG. 26 and in embodiments of the invention shown in FIGS. 27-30.

### DESCRIPTION OF THE PREFERRED EMBODIMENTS

Reference will now be made in detail to the preferred embodiments of the invention, examples of which are illustrated in the accompanying drawings. Wherever possible, the same reference numbers will be used throughout the drawings to refer to the same or like parts. The following general description applies to preferred embodiments of the present invention.

The present invention comprises of an endoscope sheath (ES) and a rear view module. The endoscope sheath (ES) is a hollow tubular structure which can be made of plastic, rubber, vinyl or any other suitable material that is preferably pliable. The endoscope sheath (ES) is sized to fit over a corresponding endoscope. The endoscope could be one of many kinds such as colonoscope, gastroscope, cystoscope, hysteroscope, laproscope etc. Means is provided to pull the endoscope sheath (ES) over corresponding endoscope. The endoscope sheath (ES) is preferably of a disposable kind to prevent cross infection and contamination among patients. The rear view module is of rectangular, square, tubular, discoid or of any other suitable shape and can be made of. It is attached to endoscope sheath (ES) by a suitable mechanical articulation such as ball socket joint, hinge joint, bi-planar rolling joint etc. The rear view module consists of a rear image lens and a rear illumination bulb to obtain a rear view. The rear image lens is attached to an image processor by an electric cable. This cable transmits the image obtained by the rear image lens to the image processor. After being processed, the image is then viewed on a computer monitor or any other display unit. The rear illumination bulb is connected to a light source by an electric cable. The rear image lens and the rear illumination bulb are typically activated upon deployment of the rear view module. The rear view module is deployed using an actuator by means of cables or by other means such as spring mechanism. The rear view module is attached to the endoscope sheath (ES) such that when deployed the rear image lens faces in a direction opposite to the long axis of the endoscope and is hence able to give a rear view of the hollow organ under examination.

In the preferred embodiment of the invention, a rear instrument channel is provided. It is placed proximal to the rear view module. This channel is connected to the main instrument channel and the passage is controlled by a control valve. Typically, deployment of the rear view module opens the passage to the rear instrument channel. The rear instrument channel is used to pass surgical instruments to do various surgical procedures in areas under view of the rear image lens. It is also used to apply suction in the area under view of the rear image lens. A rear air/water channel is also provided. It is placed proximal to the rear view module. The rear air/water channel is connected to the air/water channel of the main endoscope and the passage is controlled by a valve. Typically, deployment of the rear view module opens the passage to the rear air/water channel. The rear air/water channel is used to insufflate air in the direction of view of the rear image lens for better distension and visualization. The air/water channel is also used to squirt water or air at the rear image lens and the rear illumination bulb. This enables cleaning of the rear image lens and the rear illumination bulb while still inside a hollow body organ.

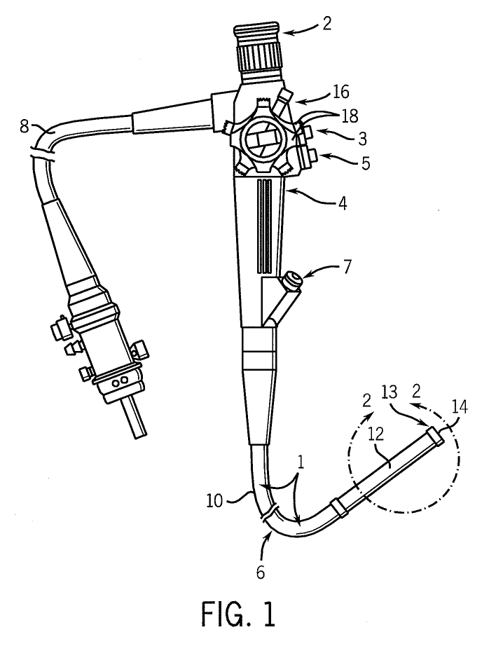

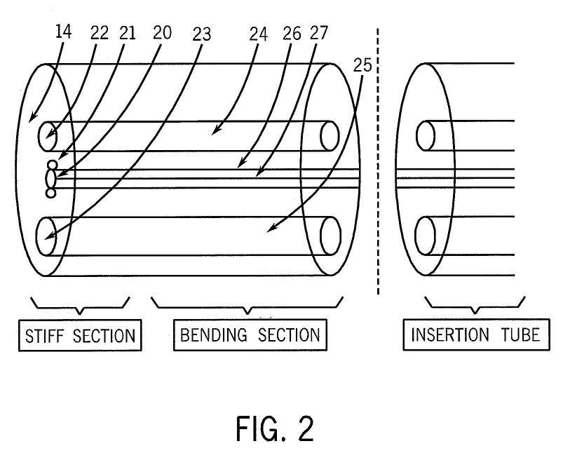

FIGS. 1 and 2 illustrate an embodiment of a conventional endoscope. It has a handle (4) from which extends a flexible shaft (1), which is inserted into a hollow organ to be inspected. The shaft consists of a proximal section (10), insertion tube (6), bending section (12) and a stiff section (13). The shaft terminates in the distal end (14), which typically houses one image lens (20), one to two illumination bulbs (21), air/water nozzle (22) and an instrument channel outlet (23). Light is transmitted from a light source through the shaft via an electric cable (26) to the illumination bulb (21). The illumination bulb illuminates the area to be examined. The image lens (20) captures images of the illuminated area. The image is then transmitted through a fiber optic cable (27) and viewed through an eyepiece (2) attached to the handle of the endoscope. Alternatively, the image is converted to a video signal and is then transmitted to an image processor by an electrical cable. The image is processed and displayed on a display unit like a computer monitor (not shown). The handle (4) of the endoscope has a grip (16) and an extension arm (8) that attaches the endoscope to a light source and an image processor. To enable the endoscope to maneuver through the turns of a hollow organ the shaft is flexible and incorporates a multitude of wires that attach the bending portion (12) with actuators (18). Typically, there are two pairs of such wires passing within the shaft, one pair for flexing the bending portion in one plane and the other pair for flexing it in an orthogonal plane. Tension is applied to these wires using the actuators (18) to move the bending portion (12) in various directions. It is also usual to provide two channels extending between the handle and the distal end of the shaft, an air/water channel (24) and an instrument channel (25). The air/water channel (24) is used to insufflate air in a hollow organ to expand it for proper visualization. The air/water channel is connected proximally to an air/water pump (not shown) and to distally to the air/water nozzle (22). It is controlled by an air/water control valve (5) located on the handle (4). The image lens (20) and the illumination bulb (21) are frequently smeared with blood, stool or other body fluids while in a hollow organ. In such a situation, the air/water channel (24) is used to squirt water or blow air at the image lens (20) and/or illumination bulb (21) in order to clean them while still inside a hollow organ. The instrument channel (25) has an instrument channel inlet (7) proximally and an instrument channel outlet (23) distally. It is used to pass surgical instruments to do various surgical procedures. It is also used to apply suction using the suction control valve (3) located on the handle (4). This suction is useful in removing fluids, air and other materials from within a hollow organ during examination.

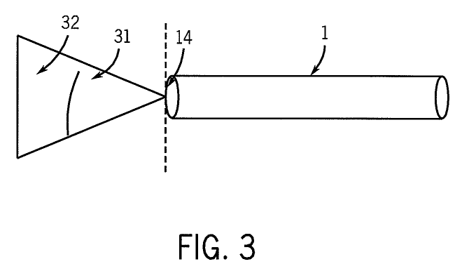

FIG. 3 illustrates the narrow field of vision (31) of about 120 degrees of a conventional endoscope (1). It also shows that conventional endoscope is only forward viewing (32).

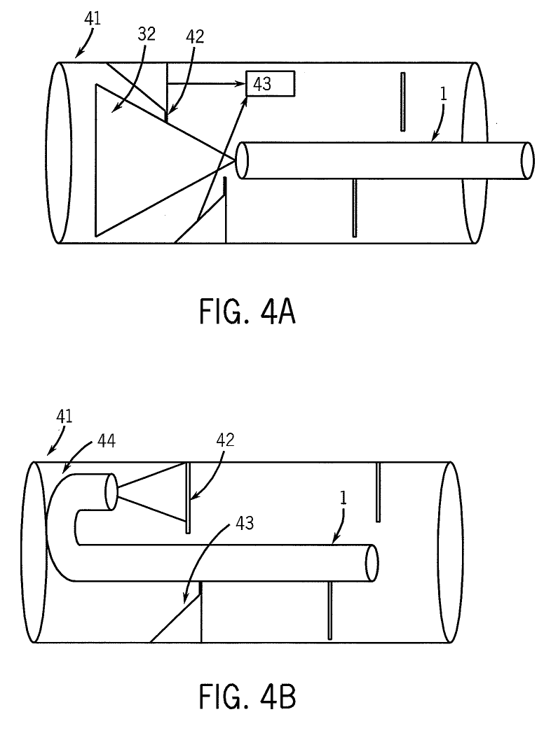

FIG. 4A shows side view of an endoscope (1) inside colon (41). The colon has mucosal folds (42). The front side of a mucosal fold blocks the view of the areas behind it during a typical endoscopic examination. These areas form the ‘blind spots’ (43) of a conventional endoscope that lie outside of the forward field of vision (32).

FIG. 4B shows side view of the retro flexion maneuver (44) of a conventional endoscope (1) inside colon (41). During this maneuver, the endoscope is advanced beyond the mucosal fold (42) to be examined. The bending portion of the endoscope is then bent to 180 degrees to visualize the rear side of a mucosal fold (43) during forward examination, the view of which is obstructed by its front side during a forward examination.

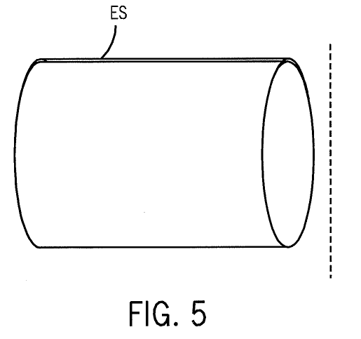

FIG. 5 shows a side view of the endoscope sheath (ES). The endoscope sheath (ES) can be made of a variety of materials such as plastic, rubber, vinyl etc. The material should preferably be pliable. Preferably, the endoscope sheath (ES) is of a disposable kind to prevent cross contamination and cross infection among patients. The endoscope sheath (ES) is sized to fit over a corresponding endoscope. Means is provided to pull the endoscope sheath (ES) over corresponding endoscope. When slipped over corresponding endoscope, the endoscope sheath leaves the front of the endoscope uncovered thus enabling the image lens and the illumination bulb of the endoscope to provide forward view. As is evident from the discussion that follows, the endoscope sheath (ES) has a ‘rear view module’ attached to it. The rear view module comprises of an illumination bulb, image lens, and electric cables connecting illumination bulb and image lens to light source and image processor respectively, and actuator cables connected to actuator to enable deployment of the rear view module. The rear view module can be of one of many shapes and sizes and can be attached to the endoscope sheath (ES) using one of many articulation means known in the prior art as illustrated in the preferred embodiments of the invention.

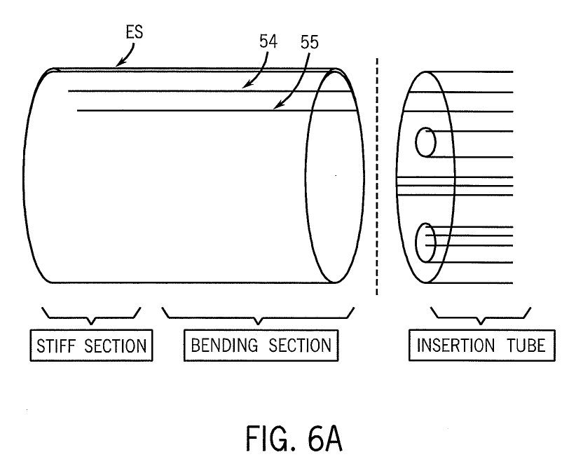

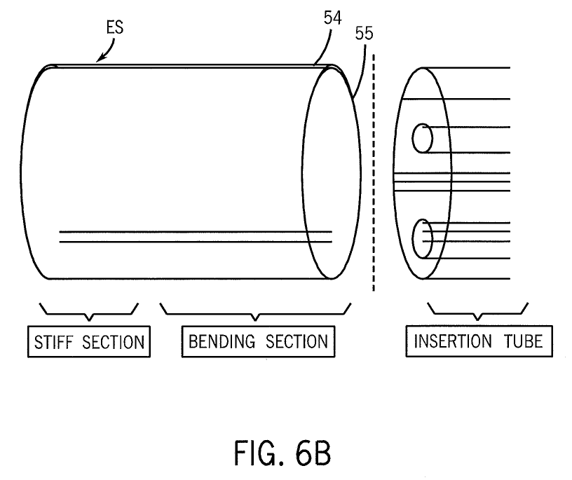

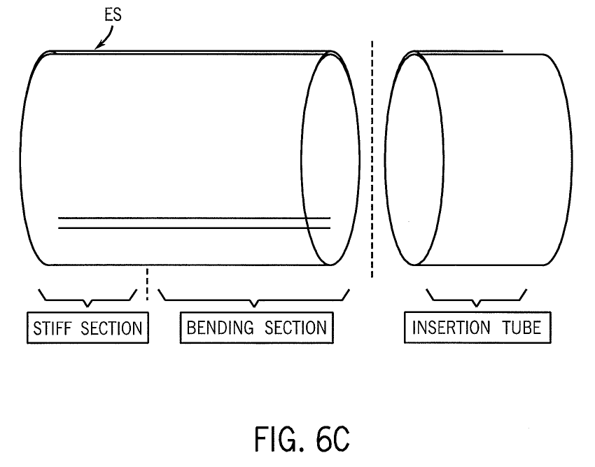

FIG. 6A shows side view of the first preferred configuration of the endoscope sheath (ES) wherein the endoscope sheath (ES) is positioned over the distal end of the endoscope. A rear view module is attached to the endoscope sheath (ES) as illustrated in the various embodiments of the invention. Rear view module comprises of an image lens and illumination bulb that is attached to an image processor and a light source respectively by means of electric cable. Additionally the rear view module is connected to an actuator by means of pull wires to enable its deployment. In this configuration, the pull wires (not shown) and electric cables (54 and 55) from the rear view module stretches over the shaft of the endoscope. This configuration has been used to illustrate the various embodiments of the endoscope sheath (ES). FIG. 6B shows side view of the second preferred configuration of the endoscope sheath (ES) wherein the endoscope sheath (ES) is positioned over the distal end of the endoscope. A rear view module is attached to the endoscope sheath (ES) as illustrated in the various embodiments of the invention. Rear view module comprises of an image lens and illumination bulb that is attached to an image processor and a light source respectively by means of electric cable. Additionally the rear view module is connected to an actuator by means of pull wires to enable its deployment. In this configuration, the pull wires (not shown) and electric cables (54 and 55) from the rear view module stretches through the instrument channel of the endoscope. FIG. 6C shows side view of the third preferred configuration of the endoscope sheath (ES) wherein the endoscope sheath (ES) is positioned over the length of the endoscope. A rear view module is attached to the endoscope sheath (ES) as illustrated in the various embodiments of the invention. Rear view module comprises of an image lens and illumination bulb that is attached to an image processor and a light source respectively by means of electric cable. Additionally the rear view module is connected to an actuator by means of pull wires to enable its deployment. In this configuration, the pull wires (not shown) and electric cables from the rear view module stretches through the endoscope sheath (ES) itself. Although the first preferred configuration has been used to illustrate the embodiments of the present invention, this should not in any way be considered limiting as the embodiments of the invention can also be carried out with the second and third configurations shown in FIGS. 6B and 6C.

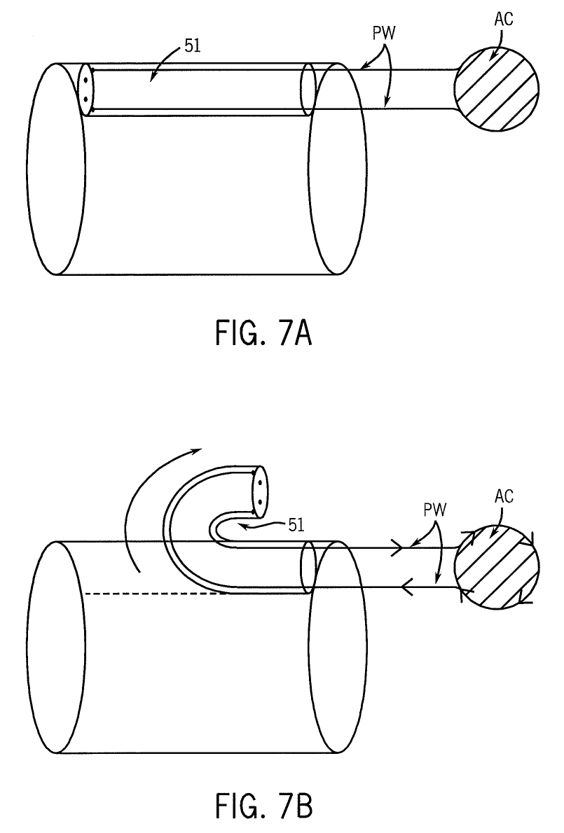

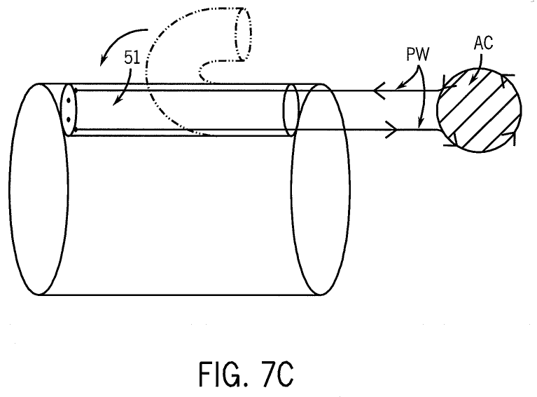

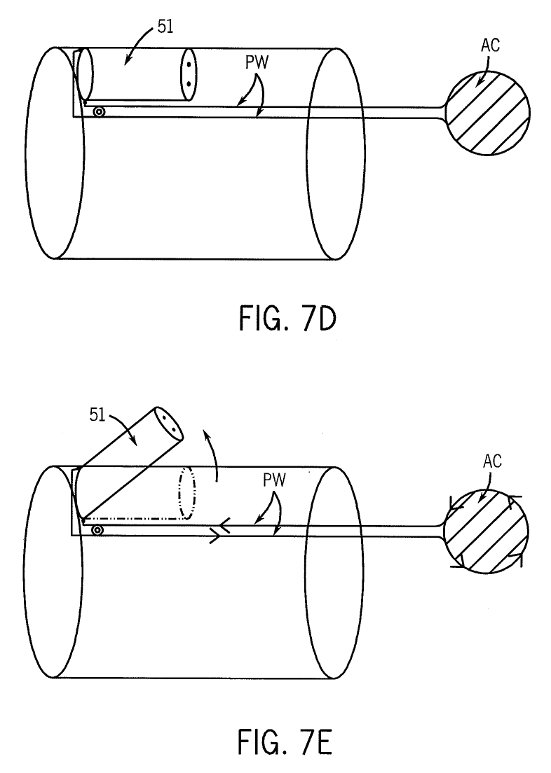

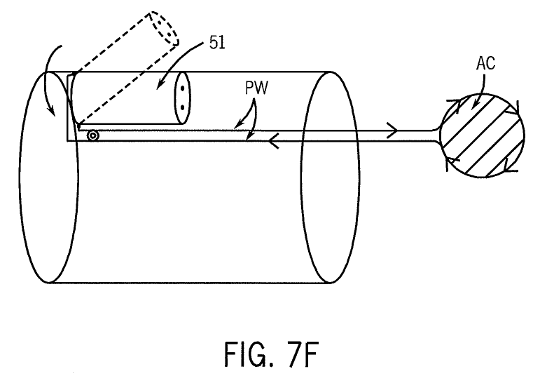

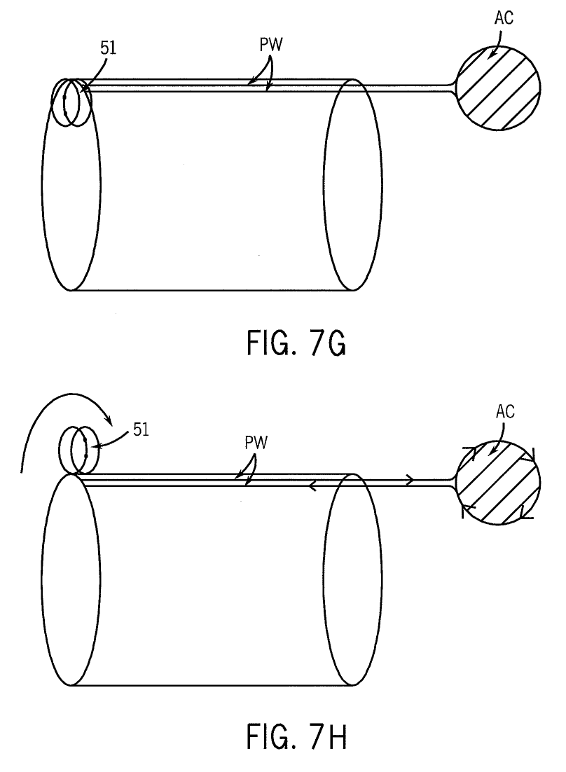

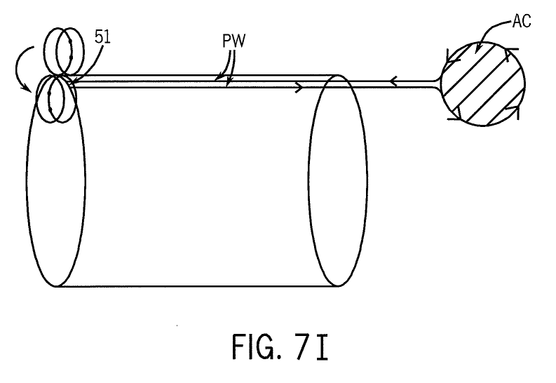

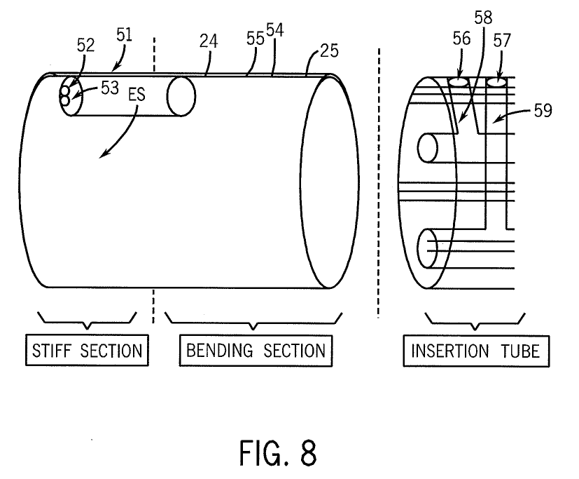

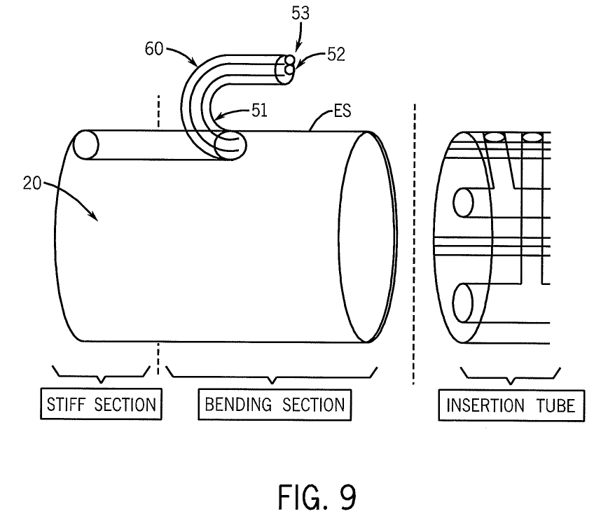

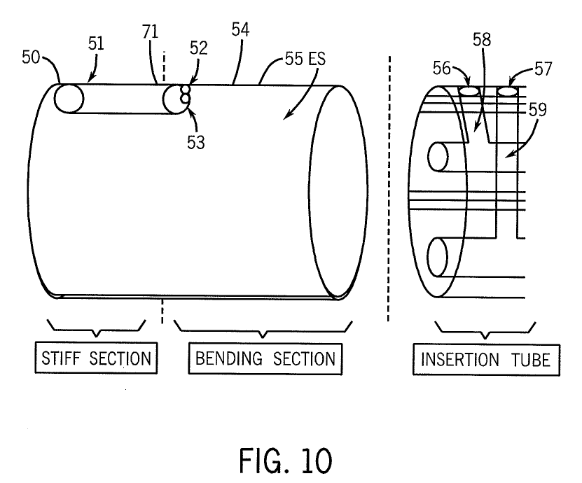

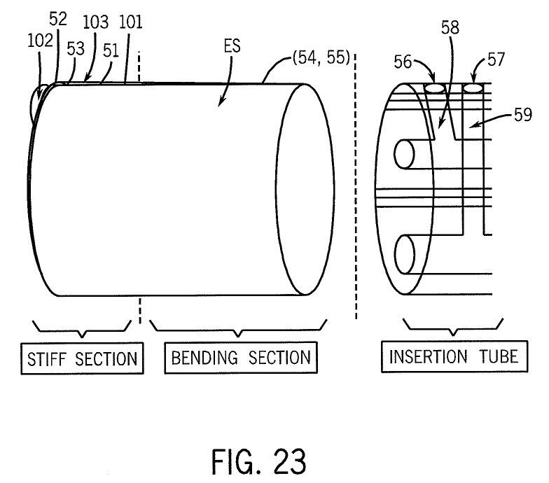

FIGS. 7A, 7B and 7C show the actuation means of the rear view module (51) when it is attached to the endoscope sheath as shown in FIGS. 8 and 9. Pull-wires (PW) are attached distally to the upper and lower part of the rear view module (51) and proximally to an actuator (AC) as shown in FIG. 7A. The rear view module (51) is in a resting position in FIG. 7A. Clock-wise movement of the actuator as shown in FIG. 7B applies tension in the top pull-wire and at the same time introduces redundancy in the lower pull-wire. The rear view module (51) is thus deployed by bending backwards on itself. Conversely, counter clock-wise movement of the actuator (AC) applies tension to the lower pull-wire and relieves tension previously applied to the upper pull-wire thus retracting the rear view module (51) back into resting position as shown in FIG. 7C. FIGS. 7D, 7E and 7F show the actuation means of the rear view module (51) when it is attached to the endoscope sheath as shown in FIGS. 10.11.14 and 15. Pull-wires (PW) are attached distally to the upper and lower part of the rear view module (51) and proximally to an actuator (AC) as shown in FIG. 7D. The rear view module (51) is in a resting position in FIG. 7D. Counter clock-wise movement of the actuator as shown in FIG. 7E applies tension in the lower pull-wire and at the same time introduces redundancy in the upper pull-wire. The rear view module (51) is thus deployed by lifting its proximal end away from the surface of the endoscope sheath. Conversely, clock-wise movement of the actuator (AC) applies tension to the upper pull-wire and relieves tension previously applied to the lower pull-wire thus retracting the rear view module (51) back into resting position as shown in FIG. 7F. FIGS. 7G, 7H and 71 show the actuation means of the rear view module (51) when it is attached to the endoscope sheath as shown in FIGS. 23, 24, 27 and 28. Pull-wires (PW) are attached distally to the outer and inner side of the rear view module (51) and proximally to an actuator (AC) as shown in FIG. 7G. The rear view module (51) is in a resting position in FIG. 7G. Clock-wise movement of the actuator as shown in FIG. 7H applies tension in the outer pull-wire and at the same time introduces redundancy in the inner pull-wire. The rear view module (51) is thus deployed by lifting it away from the front of the endoscope sheath as shown in FIG. 7H. Conversely, counter clock-wise movement of the actuator (AC) applies tension to the inner pull-wire and relieves tension previously applied to the outer pull-wire thus retracting the rear view module (51) back into resting position as shown in FIG. 71.

In the preferred embodiments of the invention and as shown in FIGS. 8, 10, 12, 14, 16, 18, 21A, 23, 27 and 29; there is a rear air/water channel (58) with a rear air/water nozzle (56) and rear instrument channel (59) with a rear instrument channel outlet (57) located on the shaft of the endoscope proximal to the position of the endoscope sheath (ES). This arrangement is feasible especially with the first and second configurations of the endoscope sheath (ES) as shown in FIGS. 6A and 6B where the endoscope sheath (ES) covers only the distal end of the endoscope. The rear air/water channel (58) provides a jet of water and a stream of air that is used to clean the rear image lens (52) and the rear illumination bulb (53) as shown in FIG. 9. It is also used to insufflate air in the field of vision of the rear image lens (52) for better distension and visualization. Surgical instruments can be passed through the rear instrument channel (59) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently.

FIG. 8 shows side view of the first preferred embodiment of the invention. The rear view module (51) is a thin tubular structure attached to the endoscope sheath (ES) by a hinge joint other suitable mechanical articulation. The distal end of the rear view module has a rear image lens (52) and a rear illumination bulb (53). The rear image lens (52) is connected to an image processor (not shown) and the rear illumination bulb (53) is connected to a light source (not shown) by electrical cables (54, 55) that run within the endoscope sheath (ES) distally and over the shaft of the endoscope proximally. Two pairs of pull wires attach the rear view module (51) to a rear view module actuator. Tension on these cables moves the rear view module (51) in vertical (or horizontal) planes for deployment and retraction. FIG. 9 shows the first preferred embodiment in FIG. 8 where the endoscope sheath (ES) is slipped over corresponding endoscope and rear view module (51) is in a retro flexed (60) position. The actuations mechanism for deployment of the rear view module is shown in FIGS. 7A, 7B and 7C. With this maneuver, the rear image lens (52) faces backward and provides a rear view. The rear illumination bulb (53) illuminates the area under view of the rear image lens (52). The main image lens of the endoscope (20) provides a front view at the same time when the rear image lens (52) is providing a rear view. However, the operator may choose to have only one view at a given time. Because the rear view module is thin, retro flexion can be achieved with a small radius of curvature and thus can be performed even inside narrow hollow organs.

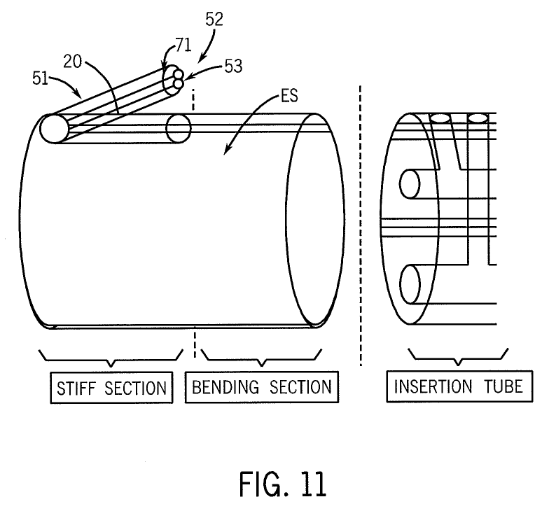

FIG. 10 shows side view of the second preferred embodiment of the present invention. The rear view module (51) is a tubular structure with a proximal end (71) and a distal end. It is attached to the distal end of the endoscope sheath (ES) by means of a suitable mechanical articulation. The rear view module (51) is flush with the outer surface of the endoscope sheath (ES).. The rear image lens (52) and the rear illumination bulb (53) are located on the proximal end (71) of the rear view module (51). The rear image lens (52) is connected to an image processor and the rear illumination bulb (53) is connected to a light source by electric cables (54, 55) running through the endoscope sheath (ES) distally and over the shaft of the endoscope proximally. The distal end of the rear view module is attached to the distal end of the endoscope sheath (ES) by a hinge joint or any other suitable mechanical articulation. The distal end (50) of the rear view module is connected to a rear view module actuator by a pair of pull wires as shown in FIG. 7C. Tension on pull wires moves the rear view module away from and towards the shaft of the endoscope sheath (ES) as shown in FIGS. 7D and 7E. FIG. 11 is a side view of the second preferred embodiment of the endoscope sheath (ES) in FIG. 10 where the endoscope sheath is slipped over the corresponding endoscope and the rear view module (51) has been deployed by lifting its proximal end (71) away from the endoscope sheath (ES) using the rear view module actuator. When fully deployed, the rear image lens (52) and the rear illumination bulb (53) face backward. The image captured by the rear image lens (52) is transmitted to an image processor. The rear illumination bulb (53) illuminates the area under view of the rear image lens (52). The main image lens (20) is able to give a forward view at the same time as the rear image lens is giving a rear view. Forward and rear view can thus be obtained simultaneously if so desired by the operator. A major advantage of this embodiment is that it makes rear view possible requiring only minimal additional space. This is of particular advantage when examining narrow body cavities.

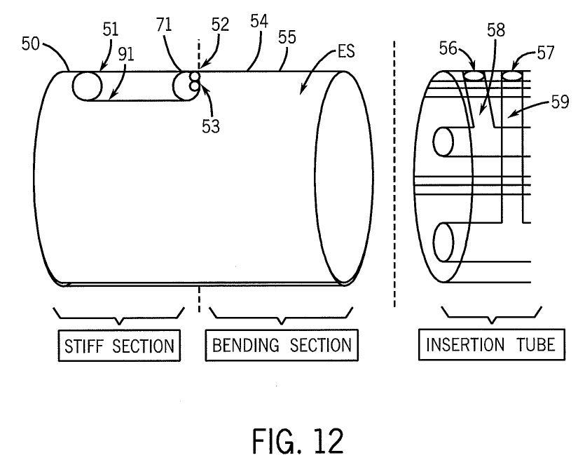

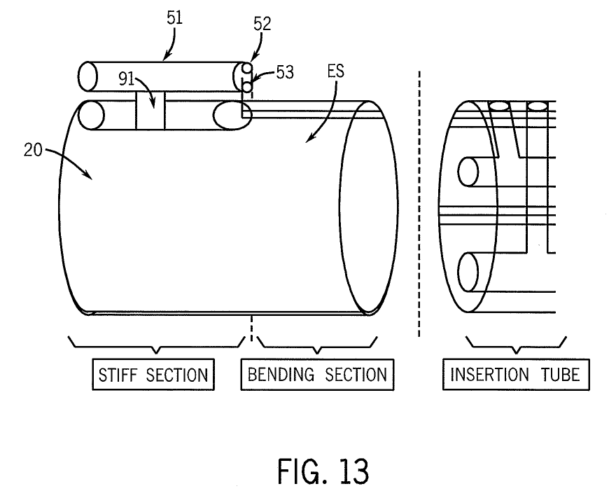

FIG. 12 shows side view of the third preferred embodiment of the invention. The rear view module (51) is a tubular structure with a proximal (71) and distal (50) end. It is preferably flush with the outer surface of the endoscope sheath (ES). The rear image lens (52) and the rear illumination bulb (53) are placed on the proximal end (71) of the rear view module (51). The rear image lens (52) is connected to an image processor and the rear illumination bulb (53) in connected to a light source by electric cables (54, 55) running through the endoscope sheath (ES) distally and over the shaft of the endoscope proximally. The rear view module (51) rests on a support pillar/spring (91) operatively connected to the endoscope sheath (ES). The support pillar/spring (91) can be extended perpendicular to the endoscope sheath (ES). The support pillar/spring is attached to a rear view module actuator by pull wires whereby tension on pull wires releases the support pillar/spring. FIG. 13 shows the third preferred embodiment of the endoscope sheath (ES) in FIG. 12 where the rear view module (51) has been deployed by releasing the support pillar/spring (91) using the rear view module actuator. In this position, the rear image lens (52) and the rear illumination bulb (53) face backward. The rear image lens (52) provides a rear view and the rear illumination bulb (53) illuminates the area under the view of the rear image lens (52). The main image lens (20) of the endoscope is able to provide a forward view at the same time when the rear image lens (52) is providing a rear view. This enables simultaneous forward and rear view if so chosen by the operator. A major advantage of this embodiment is that it provides a straight rear view that is desirable for certain surgical procedures.

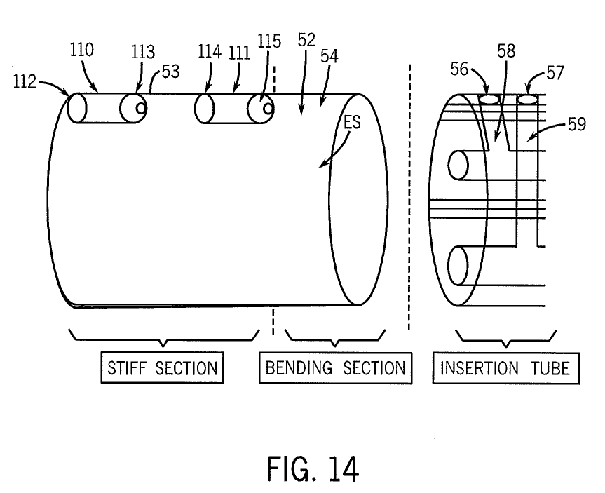

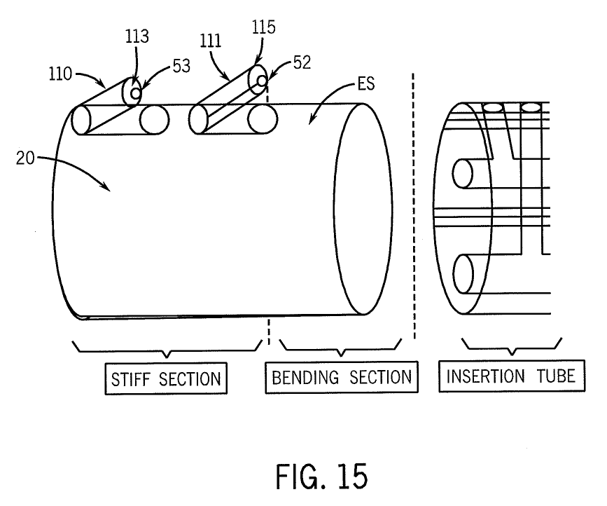

FIG. 14 shows a side view of the fourth preferred embodiment of the endoscope sheath (ES). The rear view module (51) is made of two sub modules, the rear image module (111) and the rear illumination module (110). The sub modules are small tubular structures and are preferably flush with the outer surface of the endoscope sheath (ES). The rear image module (111) contains the rear image lens (52) and the rear illumination module contains the rear illumination bulb (53). The rear image lens (52) is placed on the proximal end (115) of the rear image module (111) and the rear illumination bulb (53) is placed on the proximal end (113) of the rear illumination module (110). The rear image lens (52) is connected to an image processor by an electric cable (54) and the rear illumination bulb (53) is connected to a light source by an electric cable (55) running through the endoscope sheath (ES) distally and over the shaft of the endoscope proximally. The distal end (114) of the rear image module and the distal end (112) of the rear illumination module are attached to the endoscope sheath (ES) by a hinge joint or any other suitable mechanical articulation. The distal ends of the rear image module and of the rear illumination module (112, 114) are connected to rear module actuators by pull wires. Tension on pull wires moves the rear image module (111) and rear illumination module (110) away from and towards the endoscope sheath (ES) as shown in FIG. 15. The rear image module (111) and the rear illumination module (110) are placed at optimal distance from each other. According to a variation the relative positions of the rear illumination module and the rear image module can be interchanged. According to another variation more than one rear illumination module and/or rear image module can be present. FIG. 15 is a side view of the endoscope sheath in FIG. 14 where the rear image module (111) and the rear illumination module (110) have been deployed by moving their proximal ends (113,115) away from the endoscope sheath (ES) using the rear view module actuators as shown in FIGS. 7D,7E and 7F. In this position the rear image lens (52) and the rear illumination bulb (53) face backward and provide a rear view. The main image lens (20) is able to provide a front view at the same time when the rear image lens is providing a rear view thus enabling a simultaneous front and rear view. A major advantage of the preferred embodiment is that the rear illumination module (110) can be controlled independent of the rear image module (111). This may be desirable in certain situations.

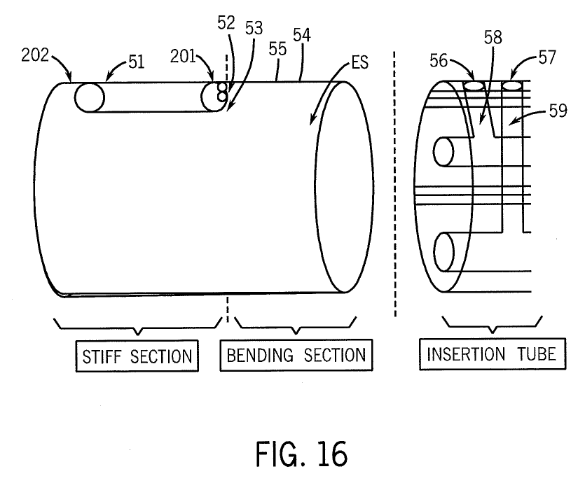

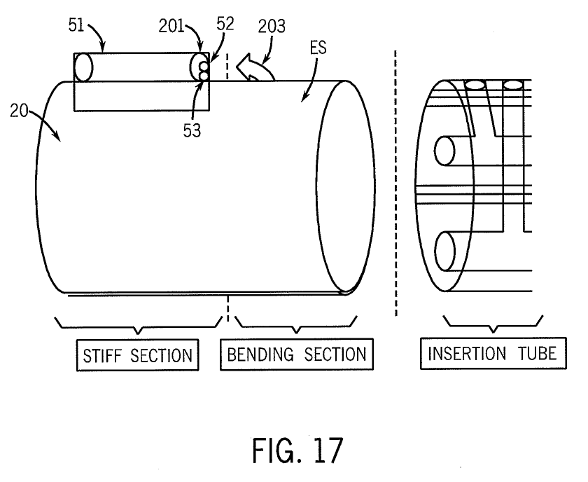

FIG. 16 shows side view of a fifth preferred embodiment of the endoscope sheath (ES). The rear view module (51) is a tubular structure with a proximal end (201) and a distal end (202). It is preferably flush with the outer surface of the endoscope sheath (ES). The rear view module (51) is connected along its length to the endoscope sheath (ES) by a hinge joint or any other suitable mechanical articulation. The rear image lens (52) and the rear illumination bulb (53) are placed on the proximal end (201) of the rear view module. The rear image lens (52) is connected to an image processor and the rear illumination bulb (53) is connected to a light source by electric cables (54, 55) running through the endoscope sheath (ES) distally and over the shaft of the endoscope proximally. Pull wires (not shown), on the outer and inner side of the rear view module, connect the rear view module to a rear view module actuator. Tension on pull wires opens and closes the module like the lid of a box (203) as shown in FIG. 17. When opened, the rear image lens (52) and the rear illumination bulb (53) face backward. The rear image lens (52) gives a rear view and the rear illumination bulb (53) illuminates the area under view of the rear image lens (52). The main image lens (20) of the endoscope is able to give a forward view at the same time as the rear image lens (52) is giving a rear view. Hence, simultaneous forward and rear view is possible if the operator so desires

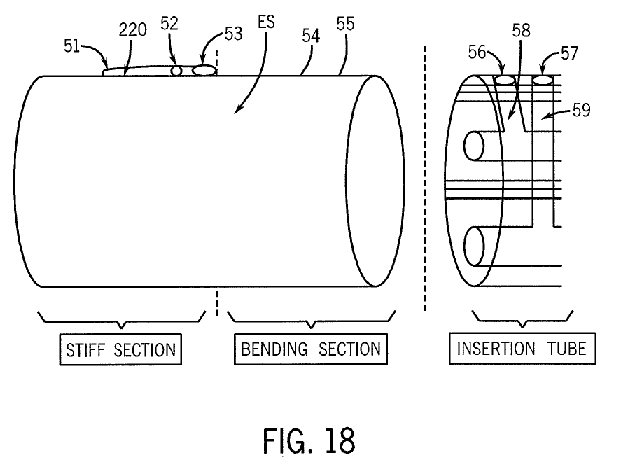

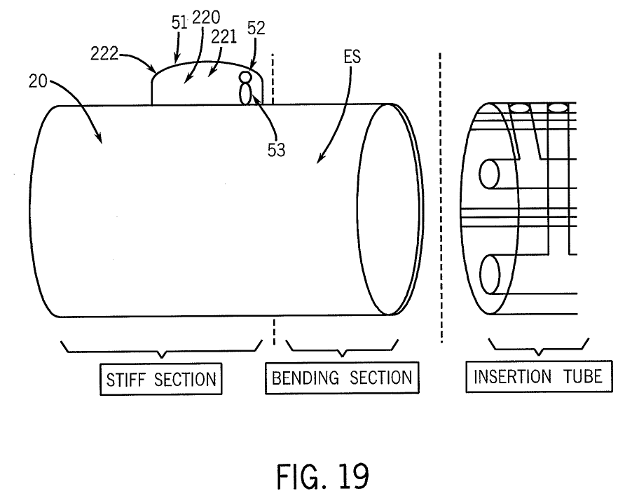

FIG. 18 shows side view of the sixth preferred embodiment of the endoscope sheath (ES). The rear view module (51) consists of an inflatable balloon (220) or any other inflatable device that is attached to the distal part of the endoscope sheath (ES). The balloon is connected to an air pump by a thin tube placed within the endoscope sheath (ES, not shown) distally and over the shaft of the endoscope proximally. When inflated, the balloon (220) has a proximal face (221) and a distal face (222) as shown in FIG. 19. The proximal face (221) of the balloon contains the rear image lens (52) and the rear illumination bulb (53). Electric cables (54, 55) connect the rear image lens (52) to an image processor and the rear illumination bulb (53) to a light source. Inflating the balloon (220) deploys the rear view module as shown in FIG. 19. When the balloon is fully inflated, the rear image lens (52) and the rear illumination bulb (53) face backwards. The rear image lens (52) gives a rear view and the rear illumination bulb (53) illuminates the area under view of the rear image lens (52). The main image lens (20) of the endoscope is able to give a forward view at the same time as the rear image lens (52) is giving a rear view. Hence, simultaneous forward and rear view is possible if the operator desires so. In a variation to the present embodiment, there can be an additional forward image lens and an additional forward illumination bulb placed on the distal face (222) of the balloon. This will widen the forward field of vision when the balloon (220) is inflated.

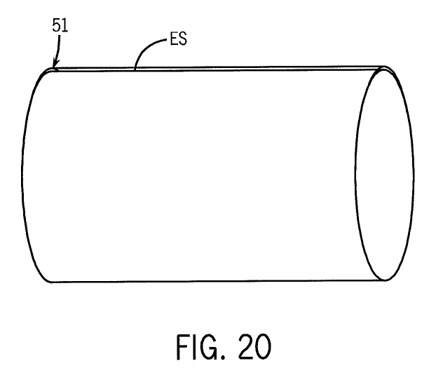

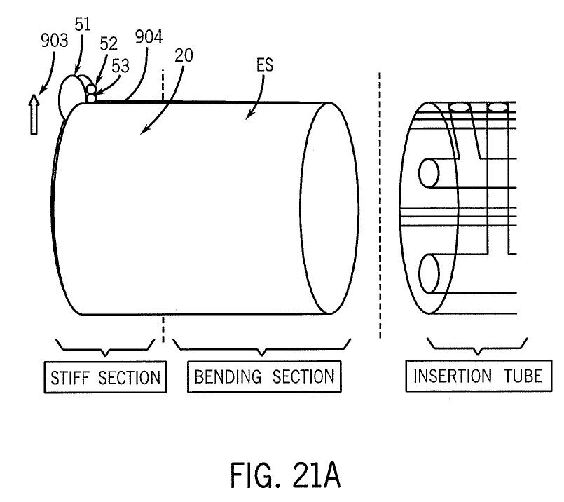

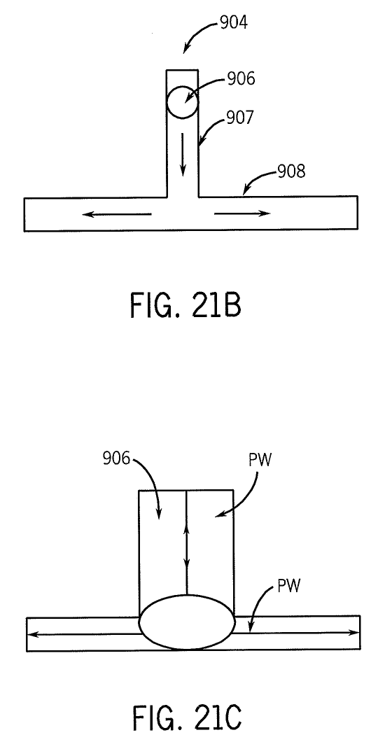

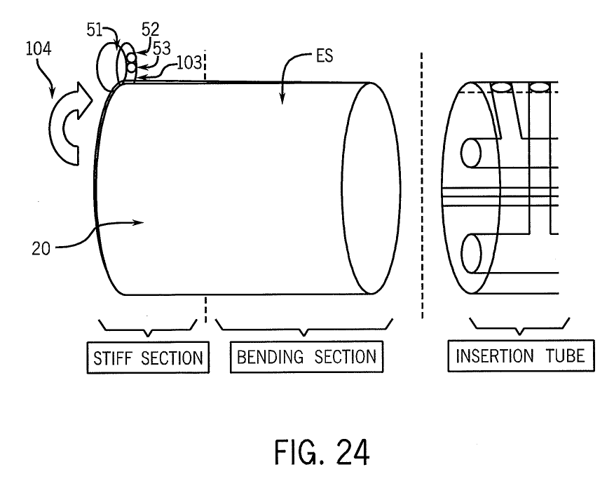

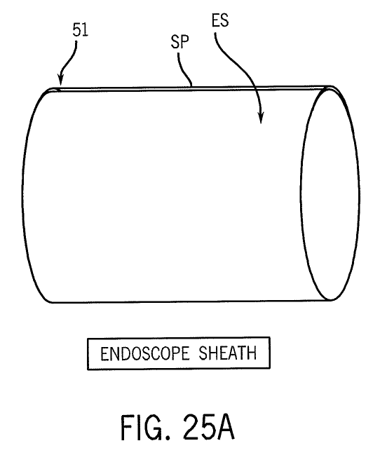

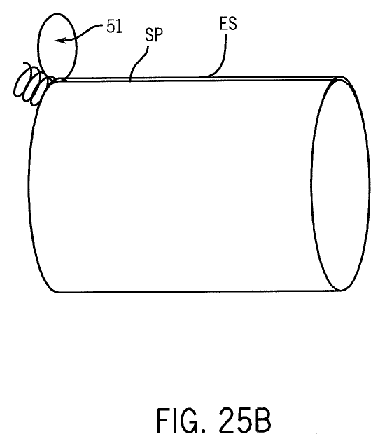

FIG. 20 show the second preferred configuration of the rear view module (51) where it is attached to the front of the endoscope sheath (ES) as illustrated in embodiments of the invention shown in FIGS. 21-24. FIGS. 7G, 7H and 71 show the actuation mechanism for deployment of the rear view module in a preferred embodiment as shown in FIGS. 23 and 24. FIGS. 25A and 25B show a spring mechanism (SP) that can alternatively be used to deploy the rear view module (51) in a preferred embodiment as shown in FIGS. 23 and 24. The mechanism of deployment of rear view module should not in any way be considered limiting.

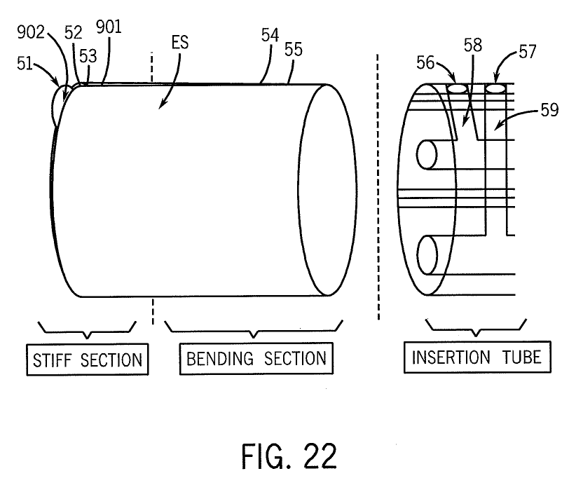

FIG. 21A shows side view of a seventh preferred embodiment of the endoscope sheath (ES). The rear view module (51) is a disc shaped structure that has a proximal face (901) and distal face (902). It is mounted on the front of the endoscope sheath (ES) by means of a suitable mechanical articulation. It comprises of a rear image lens (52) and a rear illumination bulb (53) that is placed on its proximal face (901). The rear image lens (52) is connected to an image processor and the rear illumination bulb (53) is connected to a light source by electrical cables (54, 55) running through the endoscope sheath (ES) distally and over the shaft of the endoscope proximally. In the preferred embodiment, when the endoscope sheath (ES) is slipped over the corresponding endoscope, the rear view module (51) is positioned towards the periphery of the distal end of the corresponding endoscope; but it may configured on the endoscope sheath (ES) to be positioned anywhere on the distal end of the endoscope. The proximal face (901) of the rear view module is attached to the front of the endoscope sheath (ES) by a bi-planar rolling joint (904) as shown in FIG. 21B. It allows rolling motion of the rear view module (51) in both vertical and horizontal planes from the front end of the endoscope sheath (ES). Alternatively, the rear view module may be attached using any other suitable mechanical articulation. As shown in FIG. 21B, a bi-planar rolling joint (904) consists of two grooves (907,908) placed perpendicular to each other. A small ball 1 (906) is placed within the groove. The rear view module (51) is attached to the outer part of the ball (906). As shown in FIG. 21C, pull-wires (PW) are attached to the ball vertically and horizontally. The pull-wires are attached to a rear view module actuator (not shown) proximally. The ball (906) is moved vertically by applying tension on the vertical pull-wire (PW) using rear view module actuator. Release of tension on the vertical pull-wire puts the ball (906) back in resting position as shown in FIG. 21C. While in resting position, tension of the horizontal pull-wires moves the ball (906) horizontally in left and right directions as shown in FIG. 21C.. The rear view module (51) is deployed by rolling it vertically (903) from the front of the endoscope sheath (ES), as shown in FIG. 22. Alternatively the rear view module can be deployed by rolling it horizontally from the front end of the endoscope sheath (ES). The extent of the roll is enough to move the rear image lens (52) and the rear illumination bulb (53) away from the distal end of the endoscope. In this position, the rear image lens (52) gives a rear view and the rear illumination bulb (53) illuminates the area under view of the rear image lens (52). Additionally means can be provided to rotate the rear view module (51) while deployed to widen the field of view. This may cause some distortion of the image which can be corrected by modifying the software of the image processor. The main image lens (20) of the endoscope is able to give a forward view at the same time as the rear image lens (52) is giving a rear view. Hence, simultaneous forward and rear view is possible if so desired by the operator. In a variation to the preferred embodiment the rear view module (51) may contain an additional forward image lens and an additional forward illumination bulb on its distal face (902). This will widen the forward field of view.

FIG. 23 shows side view of the eighth preferred embodiment of the endoscope sheath (ES). The rear view module (51) is a discoid structure that is mounted on the front end of the endoscope sheath (ES). It has a proximal face (101) and a distal face (102). The rear view module (51) is attached to the endoscope sheath (ES) by a hinge joint (103) or any other suitable mechanical articulation. The rear view module (51) has a rear image lens (52) and a rear illumination bulb (53) that is mounted on its distal face (102). The rear image lens (52) is connected to an image processor and the rear illumination bulb (53) is connected to a light source by electrical cables (54, 55). In resting position, when the endoscope sheath (ES) is slipped over corresponding endoscope, the rear image lens (52) and the rear illumination bulb face forward and augment the main image lens (20) and the main illumination bulb (21) to widen the forward field of view. In the preferred embodiment, the rear view module (51) is placed at the periphery of the front end of the endoscope sheath (ES) but it can be placed anywhere. The rear view module (51) is connected to a rear view module actuator by mean of pull wires as shown in FIG. 7G. Tension on these pull wires flips the rear view module (51) vertically from the front end of the endoscope sheath (ES) as shown in FIG. 7H. Alternatively, the rear view module can be flipped in a horizontal plane. FIG. 24 shows the endoscope on FIG. 23 where the rear view module (51) has been deployed by flipping it vertically (104) from the front end of the endoscope sheath (ES) while the endoscope sheath (ES) is slipped over the corresponding endoscope. In this position, the rear image lens (52) faces backward and gives a rear view. The rear illumination bulb (53) faces backward and illuminates the area under view of the rear image lens (52). The rear view module (51) can also be rotated in different directions to widen the rear field of vision. The main image lens (20) of the endoscope is able to give a forward view at the same time as the rear image lens (52) is giving a rear view. Hence, simultaneous forward and rear view is possible if the operator so desires. In a variation to the preferred embodiment, the rear view module (51) may also contain an additional forward image lens and an additional forward illumination bulb on its proximal face (101). This will increase the forward field of vision when the rear view module is deployed (104) with its proximal face (101) facing forward.

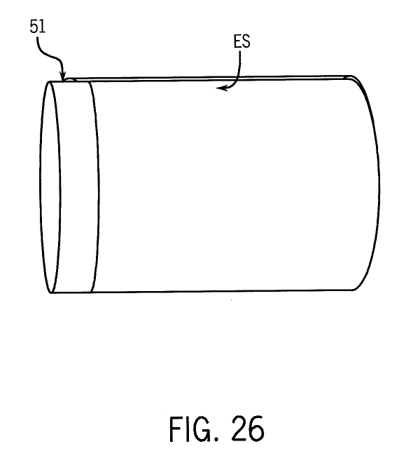

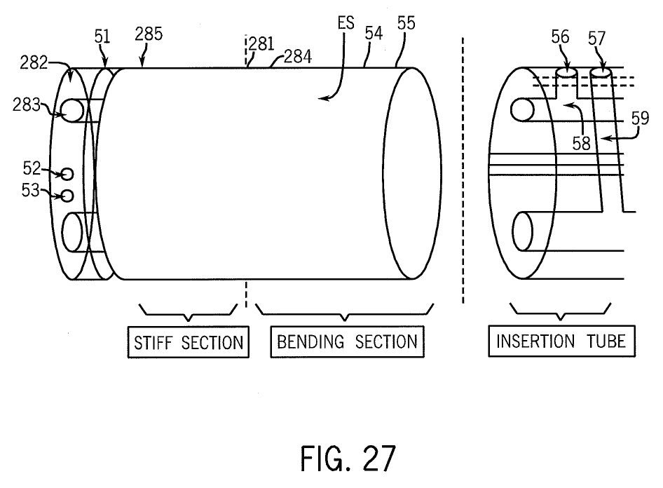

FIG. 26 show the third preferred configuration of the rear view module (51) where it is positioned on the front side of the endoscope sheath (ES) as illustrated in embodiments of the invention shown in FIGS. 27-30. The rear view module (51) can be attached to the endoscope sheath using a suitable mechanical articulation such as hinge joint.

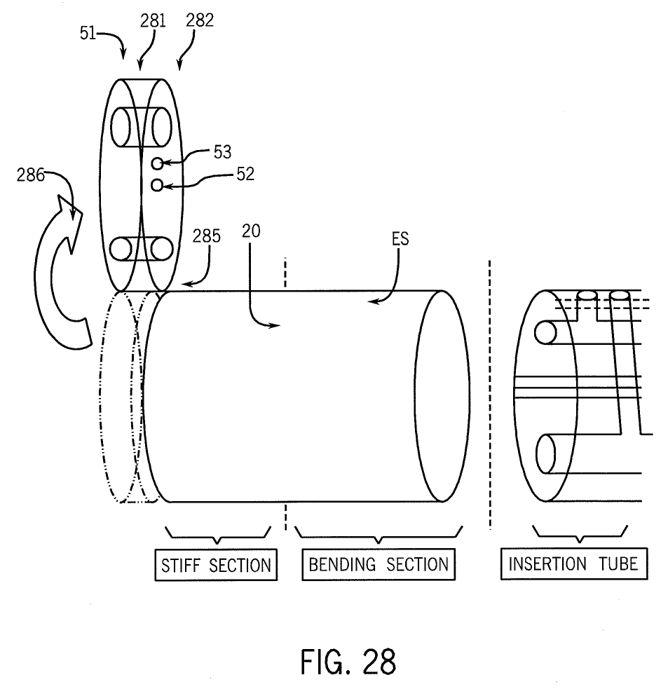

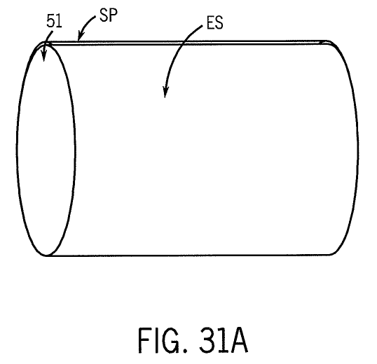

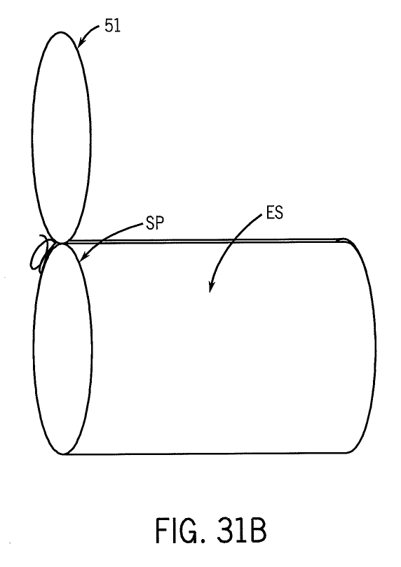

FIG. 27 shows the ninth preferred embodiment of the endoscope sheath (ES). The rear view module (51) is a discoid structure that is positioned in front of the endoscope sheath (ES). The rear view module (51) is attached to the distal end of the endoscope sheath (ES) by a hinge joint (285) or any other suitable mechanical articulation. It has a proximal face (281) and a distal face (282). The rear image lens (52) and the rear illumination bulb (53) are placed on the distal face (282) of the rear view module. The rear image lens (52) is connected to an image processor and the rear illumination bulb (53) is connected to a light source by electric cables (54, 55) running through the endoscope sheath (ES) distally and over the shaft of the endoscope proximally. In resting position and when the endoscope sheath (ES) is slipped over corresponding endoscope, the rear view module (51) covers the distal end of the endoscope and faces forward. In this position, the rear image lens (52) gives a forward view and the rear illumination bulb (53) illuminates the area in front of the endoscope. In the preferred embodiment, the diameter of the rear view module (51) is the same as that of the distal end of the corresponding endoscope. The main air/water channel (24) and the main instrument channel (25) of the endoscope extend into the rear view module (283,284). The proximal and distal face of the rear view module (281, 282) is connected to a rear view module actuator by means of pull wires. Tension on these cables flips the rear view module (51) vertically from the front of the endoscope sheath (ES) as shown in FIGS. 7G, 7H and 71. FIG. 28 shows the endoscope sheath (ES) of FIG. 27 where the endoscope sheath (ES) is slipped over the corresponding endoscope and the rear view module (51) has been deployed by flipping it vertically (286) from the front of the endoscope using the rear view module actuator. In this position, the rear image lens (52) and the rear illumination bulb (53) face backward. FIGS. 31A and 31B show a spring mechanism (SP) that can alternatively be used to deploy the rear view module (51). The rear image lens (52) gives a rear view and the rear illumination bulb (53) illuminates the area under view of the rear image lens (52). Further, means can be provided to rotate the rear view module (51) when it is in deployed position to increase the rear field of view. Upon deployment, the rear view module (51) moves away from the front of the distal end of the corresponding endoscope. It enables the main image lens (20) to give a forward view and the main illumination bulb (21) to illuminate the area in front of the distal end of the endoscope. This makes it possible to have simultaneous forward and rear view if so desired by the operator. In a variation to the preferred embodiment, the rear view module (51) may contain an additional forward image lens and an additional forward illumination bulb on its proximal face (281). When the rear view module (51) is deployed, the proximal face (281) with the additional forward image lens and additional illumination bulb faces forward and augments the main image lens (20) and the main illumination bulb (21. This widens the forward field of view when the rear view module is deployed.

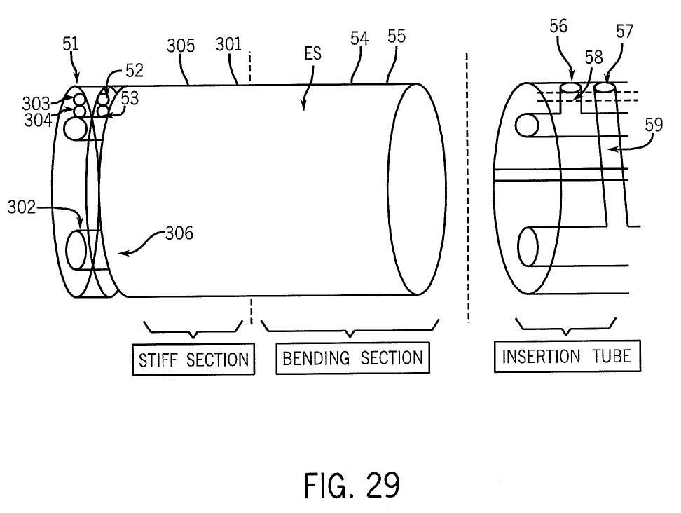

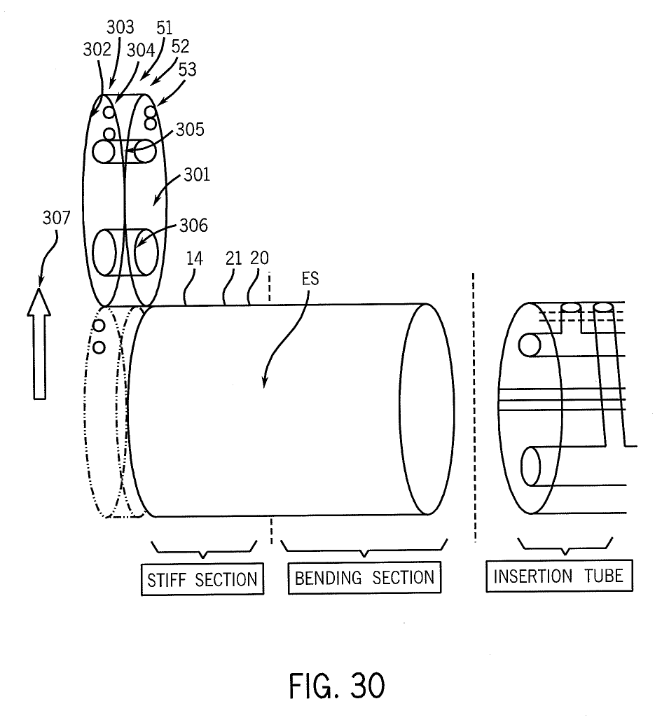

FIG. 29 shows the tenth preferred embodiment of the endoscope sheath (ES). The rear view module (51) is a discoid structure that is positioned at the front end of the endoscope sheath (ES) and attached to the distal end of the endoscope sheath (ES) by a bi-planar rolling joint as shown in FIG. 21B. Actuation means of said bi-planar joint is shown in FIG. 21C. The bi-planar joint enables movement of the rear view module (51) both vertically and horizontally relative to the front of the endoscope sheath (ES). It may also be attached by any other suitable mechanical articulation. It has a proximal face (301) and a distal face (302). The rear view module comprises of a rear image lens (52) connected to an image processor and a rear illumination bulb (53) connected to a light source by electric cables (54, 55) running through the endoscope sheath (ES) distally and over the shaft of the endoscope proximally. The rear image lens (52) and the rear illumination bulb (53) are placed on the proximal face (301) of the rear view module (51). In addition, the rear view module (51) has an additional image lens (303) and an additional illumination bulb (304) that is placed on its distal face (302). In the preferred embodiment, the diameter of the rear view module (51) is the same as that of the distal end of the endoscope corresponding to the endoscope sheath (ES). Preferably, the main air/water channel (24) and the main instrument channel (25) of the endoscope extend into the rear view module (305, 306). In resting position and when the endoscope sheath (ES) is over the corresponding endoscope, the rear view module (51) covers the image lens (20) and the illumination bulb (21) of the endoscope. In this position, the additional image lens (303) and the additional illumination bulb (304) of the rear view module faces forward and provides forward view. FIG. 30 shows the endoscope sheath (ES) of FIG. 29 where the endoscope sheath (ES) is rolled over corresponding endoscope and rear view module (51) has been deployed by sliding it vertically (307) from the front of the endoscope sheath (ES). Upon deployment, the rear image lens (52) and the rear illumination bulb (53) face backward. The rear image lens (52) gives a rear view and the rear illumination bulb (53) illuminates the area under view of the rear image lens (52). Further, means can be provided to rotate the rear view module (51) while deployed to increase the field of rear view. The rear view module (51) also moves away from front of the endoscope upon deployment. The image lens (20) is then able to give a forward view and the illumination bulb (21) is able to illuminate the area in front of the endoscope. Hence, the preferred embodiment provides simultaneous forward and rear view if so desired by the operator. The additional image lens (303) and the additional illumination bulb (304) augment the main image lens (20) and the main illumination bulb (21) and widen the forward field of vision when the rear view module (51) is deployed.

Any person/persons familiar with prior art will understand that modifications or changes to the present invention can be made without compromising its principles. Some possible variations of the present invention are; 1) the relative positions of the rear view module, rear air/water channel and the rear instrument channel may be changed; 2), more than one rear view module can be present in the endoscope sheath; 3) the shape, composition and configuration of the rear view module can be modified or changed without compromising the basic principles of the present invention; 4) the method of deployment of the rear view module can be modified without compromising the basic principles of the present invention; 5) more than one image lens and/or more than one illumination bulb can be provided in the rear view module.. The above examples are only illustrative and by no means all inclusive and these variations of the present invention should not in any way be considered limiting.

I claim:

1. An endoscopic system for examination of a hollow body component, comprising:
a removable sheath extendable about the outer periphery of a first endoscope; and
a first image lens connected to the sheath for receiving images.

2. The endoscope system of claim 1 further comprising a first endoscope received in the sheath having an outer periphery and a distal end housing a second image lens, the second image lens receiving images in a first direction.

3. The endoscope system of claim 2 wherein the sheath extends about the entire outer periphery of the first endoscope.

4. The endoscope system of claim 2 wherein the first image lens is movable between a first position and a second position wherein the first image lens receives images in a second direction at a predetermined angle to the first direction.

5. The endoscope system of claim 4 wherein the predetermined angle is 180 degrees.

6. The endoscope system of claim 4 further comprising an actuator for controlling movement of the first image lens between the first and second positions.

7. The endoscope system of claim 6 wherein the actuator includes first and second wires operatively connected to the first image lens, wherein tension on the first and second wires controls movement of the first image lens.

8. The endoscope system of claim 6 wherein the actuator includes a biasing structure in engagement with the first image lens, the biasing structure urging the first image lens towards the second position.

9. The endoscope system of claim 6 wherein the actuator includes an inflatable bladder in engagement with the first image lens, wherein inflation of the bladder urges the first image lens towards the second position.

10. The endoscope system of claim 2 wherein the first image lens is pivotable between a first storage position and a second receiving position wherein the first image lens receives images in a second direction at a predetermined angle to the first direction.

11. An endoscopic system for examination of a hollow body component, comprising:
a first endoscope having an outer periphery and a distal end housing a first image lens, the first image lens receiving images in a first direction; and
a second image lens connected to the first endoscope, the second image lens movable between a first position and a second position wherein the second image lens receives images in a second direction at an angle to the first direction.

12. The endoscope system of claim 1 1 wherein the angle is approximately 180 degrees.

13. The endoscope system of claim 11 further comprising an actuator for controlling movement of the second image lens between the first and second positions.

14. The endoscope system of claim 13 wherein the actuator includes first and second wires operatively connected to the second image lens, wherein tension on the first and second wires controls movement of the second image lens.

15. The endoscope system of claim 13 wherein the actuator includes a biasing structure in engagement with the second image lens, the biasing structure urging the second image lens towards the second position.

16. The endoscope system of claim 13 wherein the actuator includes an inflatable bladder in engagement with the second image lens, wherein inflation of the bladder urges the second image lens towards the second position.

17. The endoscope system of claim 11 wherein the second image lens is pivotable between the first position and the second position.

18. An endoscopic system for examination of a hollow body component, comprising:
a first endoscope having a distal end housing a first image lens, the first image lens receiving images in a first direction;
a sheath surrounding the first endoscope; and
a second image lens connected to the sheath, the second image lens movable between a first position and a second position wherein the second image lens receives images in a second direction at an angle to the first direction.

19. The endoscope system of claim 18 wherein the angle is approximately 180 degrees.

20. The endoscope system of claim 18 further comprising an actuator for controlling movement of the second image lens between the first and second positions.

21. The endoscope system of claim 20 wherein the actuator includes first and second wires operatively connected to the second image lens, wherein tension on the first and second wires controls movement of the second image lens.

22. The endoscope system of claim 20 wherein the actuator includes a biasing structure in engagement with the second image lens, the biasing structure urging the second image lens towards the second position.

23. The endoscope system of claim 20 wherein the actuator includes an inflatable bladder in engagement with the second image lens, wherein inflation of the bladder urges the second image lens towards the second position.

24. The endoscope system of claim 18 wherein the second image lens is pivotable between the first position and the second position.
